(19) United States
(12) Patent Application Publication
(10) Pub. No.: US 2017/0112884 A1
(43) Pub. Date: Apr. 27, 2017

(54) ENERGY COMPOSITION

(71) Applicant: Packaging and Container, LLC, Boise, ID (US)

(72) Inventors: Jesse McMullin, Riverton, UT (US); Mark Clark, South Jordan, UT (US); David Paul Millet Dawson, Cedar Hills, UT (US)

(21) Appl. No.: 15/401,960

(22) Filed: Jan. 9, 2017

Related U.S. Application Data
(63) Continuation-in-part of application No. 15/206,009, filed on Jul. 8, 2016.
(60) Provisional application No. 62/190,585, filed on Jul. 9, 2015.

(51) Int. Cl. A61K 36/17; A61K 31/137; A23L 2/52; A61K 36/185; A61K 31/194; A61K 9/00; A61K 31/522; A61K 31/475

(52) U.S. Cl. CPC A61K 36/17; A61K 31/522; A61K 31/137; A61K 31/475; A61K 36/185; A61K 31/194; A61K 9/0095; A23L 2/52; A23V 2002/00

(57) ABSTRACT

Some embodiments of the invention include administering an energy composition. In some embodiments, administering the energy composition may produce an initial perceived effect, a secondary perceived effect, and a tertiary perceived effect. The secondary perceived effect may be perceive prior to the complete dissipation of the initial perceived effect and the tertiary perceived effect may be perceive prior to the complete dissipation of the secondary perceived effect. An herb, such as Mormon tea, may be administered to produce the initial perceived effect. Caffeine may be administered to produce the secondary effect. Theobromine may be administered to produce the tertiary effect.

### CROSS-REFERENCE TO RELATED APPLICATIONS

This application is a continuation-in-part of prior patent application Ser. No. 15/206,009, filed Jul. 7, 2016, which claims priority to provisional patent application Ser. No. 62/190,585, filed Jul. 9, 2015, which are hereby incorporated by reference in their entirety.

### FIELD

The embodiments discussed herein are related to an energy composition.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

Some example embodiments generally relate to administering an energy composition to an individual. The individual may be in need of one of the perceived effects of the energy composition. The perceived effects may include, for example, perceived energy, perceived increases in energy, concentration, heartrate, and alertness. The perceived effects may also counteract, for example, fatigue, sleepiness, low motivation. The perceived effects may include physical effects including, for example, increased heart rate and sweating. The energy composition may be administered to produce such perceived effects and may be configured to produce the perceived effects in a plurality of stages. For example, administering the energy composition may produce an initial perceived effect, a secondary perceived effect, a tertiary perceived effect, etc. An overall perceived effect may include the combination of these perceived effect stages.

In some embodiments, an energy composition may comprise of one or more of the following: an herb including Mormon tea, a methylxanthine including theobromine, and/or an active ingredient including phenylethylamine. These may be configured to produce one or more of the various perceived effect stages. In some embodiments, an energy composition may reduce the delay between consumption and the perceived effects of the energy composition, which may include an energy boost or other effect, and/or stabilize the perceived increase of energy or effect, reducing the potential for a “sugar crash” or a “caffeine crash.” A “sugar crash” or “caffeine crash” may include a perceived effect dissipating in a manner that may include the perceived effect rapidly decreasing, which may include perceiving fatigue, tiredness, lack of concentration or other effects perceived with a rapid dissipation of an energy increase. A “sugar crash” may be experience without first being administered sugar.

In some embodiments, the herb may include plants or extracts from plants that produce an energy boost or effect of an energy composition. These plant or extracts may include the naturally occurring elements or synthesized/man-made elements. In various embodiments, the herb may include, for example, Mormon tea, guayusa, and/or chaparro prieto. In some embodiments, the herb may be brewed, which may reduce the delay between consumption and effect of the energy and/or may increase the effect of the energy composition. In some embodiments, a stainless steel mesh basket may be used to brew the herb for a minimum of 30 minutes and/or until the active constituents are released. In some embodiments, the herb may be configured to produce the initial perceived effect. Brewing the herb increases the potency of the perceived effect.

In some embodiments, the active ingredient may include phenylethylamine. In various embodiments, the active ingredient may also include at least one of the following: glucuronolactone, malic acid, taurine, and/or yohimbine. The active ingredients may include either naturally occurring compositions or may include synthesized/man-made compositions. For example, phenylethylamine may include natural sources such as cocoa, beans, lentils, nuts, seeds etc. and other naturally occurring sources or extracts of naturally occurring sources. Phenylethylamine may include man-made or synthesized sources such as phenylethylamine HCl and other salts or derivatives. In another example, yohimbine may include natural sources such as the yohimbe plant and extracts of such sources. Yohimbine may include man-made or synthesized sources such as yohimbine HCl and other salts or derivatives. In some embodiments, the herb may be combined with one or more of the active ingredients to produce a faster and more potent perceived effect. For example, the herb may be combined with phenylethylamine. The synergy between the herb and the active ingredient creates a faster and stronger perceived effect.

In some embodiments, the methylxanthine may include caffeine or theobromine. Other methylxanthines, including natural and made-made sources may be used in place of caffeine or theobromine. In various embodiments, the methylxanthine, including caffeine and theobromine, may be administered to produce the secondary perceived effect and the tertiary perceived effect. In some embodiments, the combination of the secondary perceived effect and the tertiary perceived effect may stabilize the effects of the energy composition, reducing the potential for a “sugar crash” or a “caffeine crash.”

In some embodiments, an energy composition may include, for example, heating water to a temperature from about 80 to 100 degrees Celsius, adding Mormon tea in an amount from about 0.000082 g/mL to about 0.0047 g/mL to the water, creating an herb and water mixture. The herb and water mixture may be, for example, removed from the heat and soaked for no shorter than 30 minutes or until active constituents are released. The herb and water mixture may be, for example, mixed with a methylxanthine including theobromine in an amount from about 0.000083 g/mL to about 0.0012 g/mL and mixed with an active ingredient including phenylethylamine in an amount from about 0.00094 g/mL to about 0.010 g/mL. The energy composition may, for example, be formulated in a predetermined volume for consumption. In other embodiments, the herb may be administered without brewing.

The method of formulating an energy composition may include, for example, adding caffeine in an amount from about 0.0028 g/mL to about 0.0050 g/mL. The method of formulating an energy composition may include, for example, adding caffeine and theobromine in an amount from about 0.0028 g/mL to about 0.0062 g/mL, coupled to stabilize the perceived effects. The method of formulating an energy composition may include, for example, adding malic acid in an amount from about 0.010 g/mL to about 0.014 g/mL. The method of formulating an energy composition may include, for example, adding yohimbine in an amount from about 0.000049 g/mL to about 0.000098 g/mL.

In some embodiments, an energy composition may comprise, for example, Mormon tea in an amount from about 0.00037 g/mL to about 0.0015 g/mL, theobromine and caffeine in an amount from about 0.0031 g/mL to about 0.0039 g/mL, and phenylethylamine in an amount from about 0.0018 g/mL to about 0.0042 g/mL.

The energy composition may further comprise, for example, water, wherein the Mormon tea is brewed in the water for no shorter than 30 minutes or until active constituents are released. The energy composition may comprise, for example, caffeine in an amount from about 0.0030 g/mL to about 0.0038 g/mL. The energy composition may comprise, for example, malic acid in an amount from about 0.010 g/mL to about 0.014 g/mL. The energy composition may comprise, for example, yohimbine in an amount from about 0.000049 g/mL to about 0.000097 g/mL.

In some embodiments, an energy composition may comprise, for example, an herb including Mormon tea in an amount from about 0.000082 g/mL to about 0.0047 g/mL, a methylxanthine including theobromine in an amount from about 0.000083 g/mL to about 0.0012 g/mL, and an active ingredient including phenylethylamine in an amount from about 0.00094 g/mL to about 0.0083 g/mL.

The energy composition may further comprise, for example, water, wherein the Mormon tea is brewed in the water for no shorter than 30 minutes or until active constituents are released. The energy composition may further comprise, for example, an herb including guayusa in an amount from about 0.00041 g/mL to about 0.0012 g/mL. The energy composition may further comprise, for example, a methylxanthine including caffeine in an amount from about 0.0028 g/mL to about 0.0050 g/mL. The energy composition may comprise, for example, caffeine and theobromine in an amount from about 0.0028 g/mL to about 0.0062 g/mL, coupled to stabilize the perceived effects. The energy composition may comprise, for example, an active ingredient including malic acid in an amount from about 0.011 g/mL to about 0.014 g/mL. The energy composition may comprise, for example, an active ingredient including yohimbine in an amount from about 0.000066 g/mL to about 0.000083 g/mL.

In some embodiments, an energy composition may comprise, for example, Mormon tea in an amount from about 0.000018 g/mL to about 0.019 g/mL, a methylxanthine including theobromine in an amount from about 0.000060 g/mL to about 0.0019 g/mL and caffeine in an amount from about 0.00083 g/mL to about 0.012 g/mL, and an active ingredient including phenylethylamine in an amount from about 0.000018 g/mL to about 0.010 g/mL.

The energy composition may further comprise, for example, water, wherein the Mormon tea is brewed in the water for no shorter than 30 minutes or until active constituents are released. The energy composition may comprise, for example, an active ingredient including malic acid in an amount from about 0.0083 g/mL to about 0.034 g/mL. The energy composition may comprise, for example, an active ingredient including yohimbine in an amount from about 0.000015 g/mL to about 0.00017 g/mL.

Additional features and advantages of the invention will be set forth in the Detailed Description which follows, and, in part, will be obvious from the description, or may be learned by the practice of the invention. The features and advantages of the invention may be realized and obtained by means of the combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages of the present invention, a more particular description of the invention will be rendered by reference to example embodiments thereof which are illustrated in a graph and a block diagram. Example embodiments of the invention will be descripted and explained with additional specificity and detail through the use of the accompanying drawings. FIGS. 1 and 2 depict only example embodiments and are not to be considered limiting of scope.

FIG. 1 is a graph that illustrates the perceived effect of some ingredients of an energy composition according to some embodiments.

FIG. 2 illustrates a block diagram of an example method for producing an energy composition according to some embodiments.

### DETAILED DESCRIPTION

Energy compositions are often consumed to experience an energy boost, increased alertness, accelerated heart rate, and/or an increased metabolism (“perceived effects”). The perceived effects of consuming an energy composition also may include a variety of perceived physical changes such as, for example, a euphoric high, increased alertness, increased focus, increased metabolism, increased heart rate, and/or any other enhanced physical change. Energy compositions may be consumed in situations where any of the perceived effects may be beneficial. Such situations may include, for example, in the morning, late at night, and/or extended periods of driving. Energy compositions have also become popular at clubs and bars, where the perceived effects may be associated with a euphoric feeling. Energy compositions may refer to any other edible or absorbable compositions such as, for example, an edible liquid, an edible powder, an edible solid, a liposomal cream, and/or any other composition suitable for delivering compositions to the body.

A possible problem associated with many commonly used energy compositions, for example, may be a perceived delay between consumption of the energy composition and the perceived effects. Another possible problem associated with many commonly used energy compositions, for example, may be the drastic reduction of the perceived effects as the energy composition wears off (“caffeine crash” and/or “sugar crash”). For example, some commonly used energy compositions' perceived effects have a minimal lasting effect. Also, physical shaking, or jitters, and sleep disruptions may be possible problems associated with many commonly used energy compositions. Additionally, for example, many commonly used energy compositions' perceived effects may have minimal strength.

Some embodiments of the present invention include administering an energy composition. In some embodiments, an individual seeking the perceived effects may include, for example, a person who is fatigued, groggy, in low spirits, or seeking a rush of energy. The energy composition may be configured to be administered to the individual in a manner that provides one or more of the following benefits: increased focus, increased alertness, reduce the delay between administration and the perceived effects of the energy composition, increase the strength of the perceived effects, increase the length of time the perceived effects are experienced, and/or stabilize the reduction of the perceived effects to reduce the potential for a “sugar crash” or a “caffeine crash.”

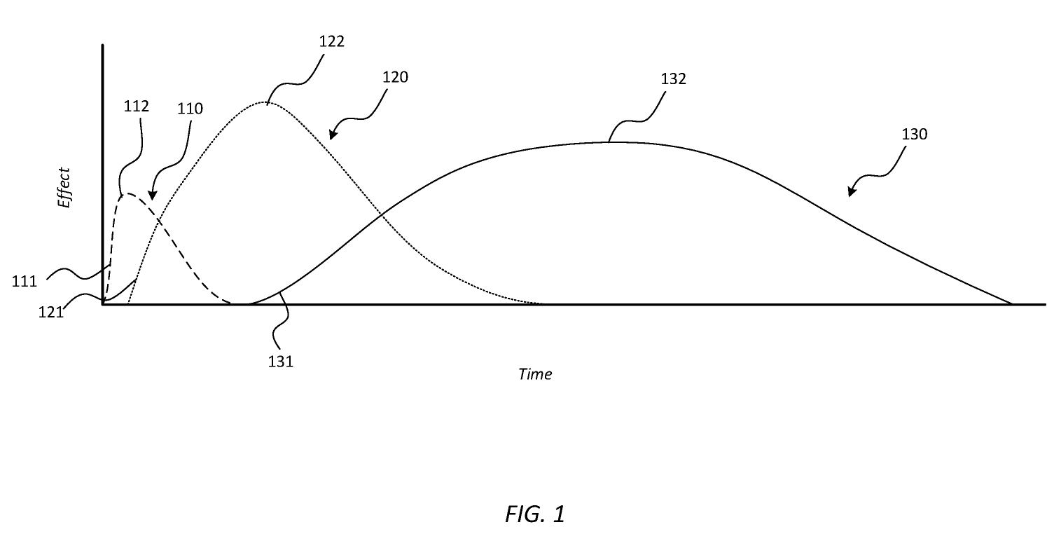

In some embodiments, the use of Mormon tea, caffeine, theobromine, and/or phenylethylamine in combination may provide one or more of the following benefits: reduce the delay between consumption and the perceived effects, increase the strength of the perceived effects, increase the length of time the perceived effects are experienced, and stabilize the reduction of the perceived effects to reduce the potential for a “sugar crash” or a “caffeine crash.” FIG. 1 is a graph of one or more perceived effects over time from administration of the energy composition as described in accordance with some embodiments. The graph shows an initial perceived effect, a secondary perceived effect, and a tertiary perceived effect. The initial perceived effect may also be referred to as the initial perceived energy increase or the first perceived energy. The secondary perceived effect may also be referred to as the secondary perceived energy increase or the second perceived energy. The third perceived effect may also be referred to as the tertiary perceived energy increase or the third perceived energy. The perceived effect of each may include any number of the previously identified perceived effects including a perceived energy boost.

The initial perceived effect may be produced by administering an herb including Mormon tea, for example. The initial perceived effect may be perceived within about 5 to 10 minutes after administration of the energy composition. An active ingredient including phenylethylamine, for example, may be combined with the herb to improve the initial perceived effect. The interaction between phenylethylamine and Mormon tea creates a synergistic effect, producing a substantially greater perceived effect. The initial perceived effect may take effect within about 5 minute. The initial perceived effect may begin to dissipate within about 25 minutes. For example, the half-life of Mormon tea may be about 15 to 20 minutes.

A secondary perceived effect may be produced by administering a methylxanthine such as caffeine, for example. The secondary perceived effect produced may take effect about 30 to 40 minutes after administration of the energy composition. The secondary perceived effect may begin to dissipate after a few hours. The half-life of caffeine, for example, may be about 2 to 5 hours.

A tertiary perceived effect may be produced by administering a methylxanthine such as theobromine, for example. The tertiary perceived effect may begin to take effect about 2 to 3 hours after administration, and may last for many hours before beginning to dissipate. For example, the half-life of theobromine may be about 7 to 12 hours.

FIG. 1 is a graph showing an example of the onset, half-life, and dissipation of the perceived effects after the administration of an example energy composition as described in accordance with some embodiments. The x-axis of FIG. 1 represents time. The y-axis of FIG. 1 represents the change in perceived effects produced from some ingredients of an example energy composition. The graph shown in FIG. 1 is not drawn to scale. Moreover, the various curves may have higher or lower maximums that what is shown in FIG. 1 and/or the duration of a curve may be extended or shrunk. FIG. 1 depicts only one example embodiment and is not intended to be limiting in scope. The various curves in the graph shown in FIG. 1 may not be drawn to scale. Line 111 shows the initial perceived effect over time. In the example depicted by FIG. 1, the initial perceived effect may be produced by administering Mormon tea. The initial perceived effect may also be produced by administering Mormon tea in combination with one or more active ingredients including yohimbine and phenylethylamine. The line 111 shows the initial perceived effect may begin to take effect, for example, about 5 or 10 minutes after administering the energy composition. The half-life of the initial perceived effect produced by Mormon tea (alone or in combination with one or more active ingredients) is shown as peak 112 in the line 111, which, for example, may be about 20 minutes after administration of the energy composition. Dissipation of the initial perceived effect is shown in the tail 110 of the line 111. As the initial perceived effect begins to dissipate (see tail 110 of line 111), the secondary perceived effect, as shown by line 121, may begin to take effect. As shown in the example depicted in FIG. 1, the initial perceived effect produced by the administration of Mormon tea (alone or in combination with one or more active ingredients) completely dissipates. However, it will be appreciated that before the initial perceived effect has completely dissipated, the secondary perceived effect has begun to take effect. In some embodiments, the secondary perceived effect may begin to take effect before the first, second, third, or fourth half-life of the initial perceived effect. The combination of the initial perceived effect and the secondary perceived effect work in synergy to create an overall perceived effect to the individual not produced by either the initial or secondary perceived effects.

The secondary perceived effect may be produced by caffeine, for example. The secondary perceived effect may begin to take effect, for example, about 30 minutes after administration of the caffeine. The half-life of the secondary perceived effect is shown as peak 122 in the line 121, which, for example, may be about 2.5 hours after administering the energy composition. Dissipation of the second perceived effect is shown in the tail 120 of line the 112. The secondary perceived effect may rapidly dissipate. As the secondary perceived effect begins to dissipate (see tail 120 of line 121), the tertiary perceived effects may begin to take effect as shown by line 131, showing the tertiary perceived effects over time. As shown in the example depicted in FIG. 1, the secondary perceived effect completely dissipates. However, it will be appreciated that before the secondary perceived effect has completely dissipated, the tertiary perceived effect has begun to take effect. In some embodiments, the tertiary perceived effect may begin to take effect before the first, second, third, or fourth half-life of the secondary perceived effect. The combination of the secondary perceived effect and the tertiary perceived effect work in synergy to create an overall perceived effect to the individual not produced by either the secondary or tertiary perceived effects.

In the example depicted in FIG. 1, the tertiary perceived effects are produced by the administration of theobromine. The tertiary perceived effects may begin to take effect, for example, about 2 hours after administering the energy composition. The half-life of the tertiary perceived effect is shown as peak 132 in the line 131, which, for example, may be about 7 hours after ingestion of the energy composition. As shown in the example depicted in FIG. 1, the tertiary perceived effect completely dissipates. Due to, in part, to a long half-life, the tertiary perceived effect dissipates slowly enough to avoid a “sugar crash” or “caffeine crash.”

Although the secondary perceived effects of caffeine may not take a long time to take effect, the addition of the initial perceived effect, for example, may reduce the delay between administration of the energy composition and a desired perceived effect. The initial perceived effect may be configured to provide this quick and potent effect. In some embodiments, the initial perceived effect may be increased in potency by brewing the herb and may be increasingly strong proportional to the amount of time the herb is brewed. Additionally, the initial perceived effect may be configured for a stronger potency by administering the herb in combination with one or more active ingredients. The combination of the one or more active ingredients creates a synergistically stronger initial perceived effect by increasing the potency of the initial perceived effect produced by the herb alone.

After the first half-life of the secondary perceived effect is reached and/or begins to dissipate, people generally may feel a “caffeine crash” because the perceived effects, for example, may wear off rapidly. However, the addition of the tertiary perceived effect may stabilize the reduction of the overall perceived effect to reduce the potential for a “sugar crash” or “caffeine crash.” Additionally, the tertiary perceived effects, for example, although more potent, may be significantly slower than caffeine, which may prevent the tertiary perceived effect from wearing off rapidly. The tertiary perceived effect's slow onset and long-lasting perceived effect, for example, may counter the secondary perceived effect's rapid onset and dissipation. The tertiary perceived effect, for example, may increase the length of time the perceived effects are experienced and/or reduce the potential for a “sugar crash” or “caffeine crash.”

The combination of the initial perceived effect, the secondary perceived effect, and the tertiary perceived effect creates a synergistic effect greater than each part individually. The initial perceived effect may provide a rapid and potent effect on the individual. The secondary initial effect may counteract the dissipation of the initial perceived effect. The tertiary perceived effect may counteract the dissipation of the secondary effect and further provide a longer and more stable overall perceived effect to the individual. Administering an energy composition configured with the initial, secondary, and tertiary perceived effects may provide a rapid onset of desired perceived effects, including a strong and potent energy boost, that is stable and maintained over a long period of time.

In some embodiments, the time release energy and focus (i.e. the initial, secondary, and tertiary perceived effects) may be produced by administering an energy composition that produces time released energy. The human body may metabolize the energy composition through a plurality of organs. For example, the active ingredient, including phenylethylamine and yohimbine, along with some of the phytonutrients of the herb including Mormon tea, may cross the blood-brain barrier. In such an embodiment, crossing the blood-brain barrier may produce a very fast and strong perceived energy increase. The embodiment may further include, for example, the methylxanthine including caffeine and metabolites of the Mormon Tea being processed in the liver, which may result in the methylxanthine including caffeine metabolizing in the liver. The metabolizing of the caffeine in the liver may result in theobromine being produced in the liver. The metabolism of theobromine in the liver may not prevent a “caffeine crash.” The embodiment may further include, for example, a methylxanthine including theobromine. Administering the energy combination with the methylxanthine including theobromine may include the methylxanthine including theobromine being metabolized through the kidney. The combination of administering an energy composition that includes the herb including Mormon tea, the methylxanthine including caffeine, and the methylxanthine including theobromine may create a synergistic effect in which the time released energy produces a fast initial perceived energy increase that may last for many hours without a “caffeine crash.” The perceived effect from administering the energy composition including Mormon tea, the methylxanthine including caffeine, and the methylxanthine including theobromine may be greater than administering each component individually. The energy composition may further include the combination of yohimbine, phenylethylamine, and malic acid, which may further produce a synergistic effect greater than administering each component individually.

In some embodiments, the method for improving alertness may include an individual in need of an overall perceived energy increase. The method may include producing an initial perceived energy increase within about 10 minutes by administering an herb including Mormon tea in an amount from about 0.00037 g/mL to about 0.0015 g/mL followed by producing a secondary perceived energy increase within about 40 minutes by administering a methylxanthine including caffeine in an amount from about 0.0030 g/mL to about 0.0038 g/mL, followed by producing a tertiary perceived energy increase within about 135 minutes by administering a methylxanthine including theobromine in an amount from about 0.000083 g/mL to about 0.0012 g/mL. The secondary perceived energy increase may be perceived prior to the complete dissipation of the initial perceived energy increase and the tertiary perceived energy increase may be perceived prior to the complete dissipation of the secondary perceived energy increase. The various stages of the perceived energy increases may include time released energy to the body of the individual. The time released energy may include the perceived effects previously discussed, may also include, for example, other effects such as increased heart rate, focus, concentration, or sweating. The various stages of the perceived energy increases (i.e. the initial, secondary, and tertiary perceived effects) may be produced by administering one energy composition. Administering one energy composition that produces the various stages of the perceived energy increases avoids the need to administer separate energy compositions. Moreover, administering one energy composition that includes the combination of the herb, the active ingredient, and the methylxanthines, creates a synergistic effect. For example, the energy composition may be administered including Mormon tea, caffeine, and theobromine. In combination, these compositions may produce a time released energy effect far greater than any one of the compositions administered alone may produce. In another example, the energy composition may be administered including Mormon tea, phenylethylamine, yohimbine, caffeine, theobromine, and malic acid. In combination, these compositions may produce a time released energy effect far greater than any one of the compositions administered alone may produce.

In other embodiments, administering the energy composition may include administering the energy composition with an active ingredient including phenylethylamine, in an amount from about 0.0018 g/mL to about 0.0042 g/mL. The active ingredient including phenylethylamine in combination with the herb including Mormon tea produces a synergistically stronger perceived effect than either administer alone. The phenylethylamine may produce the initial energy increase.

In other embodiments, administering the energy composition may include administering the energy composition with yohimbine in an amount from about 0.000049 g/mL to about 0.000097 g/mL. The yohimbine may produce the initial energy increase.

In other embodiments, administering the energy composition may include administering the energy composition with an active ingredient further includes administering malic acid in an amount from about 0.010 g/mL to about 0.014 g/mL.

In other embodiments, administering the energy composition may include administering the energy composition with the herb including Mormon tea after the herb is brewed in water for no shorter than 30 minutes or until active constituents are released.

In some embodiments, the method for improving alertness may include an individual desiring an increase in overall perceived energy increase and producing the overall perceived energy increase by administering an energy composition. The energy composition may produce an initial, secondary, and tertiary perceived energy increase. The initial perceived energy increase may be perceived within about 5 minutes by administering an herb including Mormon tea in an amount from about 0.000082 g/mL to about 0.0047 g/mL in combination with an active ingredient including phenylethylamine in an amount from about 0.00094 g/mL to about 0.0083 g/mL. The secondary perceived energy increase may be perceived within about 30 minutes by administering a methylxanthine including caffeine in an amount from about 0.0028 g/mL to about 0.0050 g/mL. The tertiary perceived energy increase may be perceived within about 120 minutes by administering a methylxanthine including theobromine in an amount from about 0.000083 g/mL to about 0.0012 g/mL. The secondary perceived energy increase may be perceived prior to the complete dissipation of the initial perceived energy increase and the tertiary perceived energy increase may be perceived prior to the complete dissipation of the secondary perceived energy increase. The various stages of the perceived energy increases may include time released energy to the body of the individual. The time released energy may include the perceived effects previously discussed, may also include, for example, other effects such as increased heart rate, focus, concentration, or sweating. The various stages of the perceived energy increases (i.e. the initial, secondary, and tertiary perceived effects) may be produced by administering one energy composition. Administering one energy composition that produces the various stages of the perceived energy increases avoids the need to administer separate energy compositions. Moreover, administering one energy composition that includes the combination of the herb, the active ingredient, and the methylxanthines, creates a synergistic effect. For example, the energy composition may be administered including Mormon tea, caffeine, and theobromine. In combination, these compositions may produce a time released energy effect far greater than any one of the compositions administered alone may produce. In another example, the energy composition may be administered including Mormon tea, phenylethylamine, yohimbine, caffeine, theobromine, and malic acid. In combination, these compositions may produce a time released energy effect far greater than any one of the compositions administered alone may produce.

In other embodiments, administering the energy composition may include administering the energy composition with an active ingredient including yohimbine in an amount from about 0.000066 g/mL to about 0.000083 g/mL. The yohimbine may produce the initial energy increase.

In other embodiments, administering the energy composition may include administering the energy composition with an active ingredient including malic acid in an amount from about 0.011 g/mL to about 0.014 g/mL.

In other embodiments, administering the energy composition may include administering the energy composition with an herb including guayusa in an amount from about 0.00041 g/mL to about 0.012 g/mL. The guayusa may produce the secondary energy increase.

The energy composition may be administered in any number of combinations of amounts of the herb, the active ingredient and the methylxanthine. The ranges of amounts of the individual components of the energy composition may vary according the ranges discussed herein. The use of the measurement of “g/mL” does not in any way limit the energy composition to liquids. The energy composition may be administered in various dry forms or other forms, including through the delivery systems discussed herein. Additionally, the components of the energy composition may be measured in ratios of components.

In one embodiment, a method for increasing energy in a subject can include: providing the subject, wherein the subject has an initial perceived energy level, and wherein the subject has a desire for an increased perceived energy level that is greater than the initial perceived energy level; and administering a composition to the subject, the composition having Mormon tea in an amount from about 0.00037 g/mL to about 0.0015 g/mL that provides a first perceived energy and a first methylxanthine in an amount from about 0.0030 g/mL to about 0.0038 g/mL that provides a second perceived energy and a second methylxanthine in an amount from about 0.000083 g/mL to about 0.0012 g/mL that provides a third perceived energy. In one aspect, the composition provides the first perceived energy having a first perceived energy increase within about 10 minutes of administering the composition from the herb including Mormon tea, wherein then the first perceived energy has a first perceived energy peak and then a first perceived energy decline In one aspect, the composition provides the second perceived energy having a second perceived energy increase within about 40 minutes of administering the composition from the first methylxanthine, wherein then the second perceived energy has a second perceived energy peak and then a second perceived energy decline. In one aspect, the composition provides the third perceived energy having a second perceived energy increase within about 135 minutes of administering the composition from the second methylxanthine, wherein then the third perceived energy has a third perceived energy peak and then a third perceived energy decline. In one aspect, the first perceived energy increase is perceived by the subject before the second perceived energy increase and the third perceived energy increase is perceived by the subject after the second perceived energy increase, and wherein during the first perceived energy decline the second perceived energy increase become greater than the first perceived energy such that the subject perceives the second perceived energy increase prior to complete dissipation of the first perceived energy, and wherein during the second perceived energy decline the third perceived energy increase become greater than the second perceived energy such that the subject perceives the third perceived energy increase prior to complete dissipation of the second perceived energy.

In one aspect, the first methylxanthine includes caffeine and the second methylxanthine includes theobromine. In one aspect, the composition includes phenylethylamine in amount from about 0.0018 g/mL to about 0.0042 g/mL that contributes to providing the first perceived energy. In one aspect, the composition includes yohimbine in an amount from about 0.000049 g/mL to about 0.000097 g/mL that contributes to providing the first perceived energy. In one aspect, the composition includes phenylethylamine in amount from about 0.0018 g/mL to about 0.0042 g/mL and yohimbine in an amount from about 0.000049 g/mL to about 0.000097 g/mL, wherein the phenylethylamine and yohimbine both contribute to providing the first perceived energy. In one aspect, at least one of the Mormon tea, first methylxanthine or second methylxanthine is provided in the composition as an extract of a plant. In one aspect, the composition includes malic acid in an amount from about 0.010 g/mL to about 0.014 g/mL. In one aspect, the composition includes guayusa in an amount from about 0.00041 g/mL to about 0.012 g/mL.

In one aspect, the herb including Mormon tea is brewed in water for no shorter than 30 minutes or until active constituents are released before being formulated into the composition.

In one aspect, wherein prior to administering the composition, the subject has an initial alertness level, and wherein the subject has a desire for an increased alertness level that is greater than the initial alertness level, wherein the first perceived energy, second perceived energy, and third perceived energy provide the increased alertness. In one aspect, prior to administering the composition, the subject feels fatigued, wherein the first perceived energy, second perceived energy, and third perceived energy reduce the feeling of being fatigued. In one aspect, prior to administering the composition, the subject has an initial mental concentration level, and wherein the subject has a desire for an increased mental concentration level that is greater than the initial alertness level, wherein the first perceived energy, second perceived energy, and third perceived energy provide the increased mental concentration. In one aspect, the subject does not perceive energy lower than the initial perceived energy level until after complete dissipation of the third perceived energy. In one aspect, the subject does not feel a need to increase their perceived energy level until during or after the third perceived energy decline. In one aspect, the subject does not perceive an energy crash until after complete dissipation of the third perceived energy.

In one embodiment, the administering of the composition is in an oral bolus consumed orally substantially within about 1 second, or within about 2 seconds, or within about 3 seconds, or within about 4 seconds, or within about 5 seconds, or within about 10 seconds, or within about 20 seconds, or within about 30 seconds, or within about 1 minute. In one aspect, the composition is a liquid beverage that is administered orally.

In one embodiment, a method for increasing energy in a subject can include: providing the subject, wherein the subject has an initial perceived energy level, and wherein the subject has a desire for an increased perceived energy level that is greater than the initial perceived energy level, and wherein the subject has a desire for sustained perceived energy; and administering a composition to the subject. In one aspect, the composition the composition can include: Mormon tea in an amount from about 0.00037 g/mL to about 0.0015 g/mL and phenylethylamine in amount from about 0.0018 g/mL to about 0.0042 g/mL, the Mormon tea and phenylethylamine cooperatively provides a first perceived energy; a first methylxanthine including caffeine in an amount from about 0.0030 g/mL to about 0.0038 g/mL that provides a second perceived energy; and a second methylxanthine including theobromine in an amount from about 0.000083 g/mL to about 0.0012 g/mL that provides a third perceived energy. In one aspect, the composition provides the first perceived energy having a first perceived energy increase within about 10 minutes of administering the composition from the herb including Mormon tea, wherein then the first perceived energy has a first perceived energy peak and then a first perceived energy decline. In one aspect, the composition provides the second perceived energy having a second perceived energy increase within about 40 minutes of administering the composition from the first methylxanthine, wherein then the second perceived energy has a second perceived energy peak and then a second perceived energy decline. In one aspect, the composition provides the third perceived energy having a second perceived energy increase within about 135 minutes of administering the composition from the second methylxanthine, wherein then the third perceived energy has a third perceived energy peak and then a third perceived energy decline. In one aspect, the first perceived energy increase is perceived by the subject before the second perceived energy increase and the third perceived energy increase is perceived by the subject after the second perceived energy increase, and wherein during the first perceived energy decline the second perceived energy increase become greater than the first perceived energy such that the subject perceives the second perceived energy increase prior to complete dissipation of the first perceived energy, and wherein during the second perceived energy decline the third perceived energy increase become greater than the second perceived energy such that the subject perceives the third perceived energy increase prior to complete dissipation of the second perceived energy so as to provide the sustained perceived energy. In one aspect, the composition includes yohimbine in an amount from about 0.000049 g/mL to about 0.000097 g/mL that contributes to providing the first perceived energy.

In one embodiment, a method for increasing mental performance in a subject can include: providing the subject, wherein the subject has an initial perceived energy level for an initial mental performance, and wherein the subject has a desire for an increased perceived energy level for an increased mental performance that is greater than the initial mental performance; and administering a composition to the subject. In one aspect, the composition can include: Mormon tea in an amount from about 0.00037 g/mL to about 0.0015 g/mL and phenylethylamine in amount from about 0.0018 g/mL to about 0.0042 g/mL, the Mormon tea and phenylethylamine cooperatively provides a first perceived energy; a first methylxanthine including caffeine in an amount from about 0.0030 g/mL to about 0.0038 g/mL that provides a second perceived energy; and a second methylxanthine including theobromine in an amount from about 0.000083 g/mL to about 0.0012 g/mL that provides a third perceived energy. In one aspect, the composition provides the first perceived energy having a first perceived energy increase within about 10 minutes of administering the composition from the herb including Mormon tea, wherein then the first perceived energy has a first perceived energy peak and then a first perceived energy decline, wherein the first perceived energy correlates with a first mental performance. In one aspect, the composition provides the second perceived energy having a second perceived energy increase within about 40 minutes of administering the composition from the first methylxanthine, wherein then the second perceived energy has a second perceived energy peak and then a second perceived energy decline, wherein the second perceived energy correlates with a second mental performance. In one aspect, the composition provides the third perceived energy having a second perceived energy increase within about 135 minutes of administering the composition from the second methylxanthine, wherein then the third perceived energy has a third perceived energy peak and then a third perceived energy decline, wherein the third perceived energy correlates with a third mental performance. In one aspect, the first perceived energy increase is perceived by the subject by increased mental performance before the second perceived energy increase is perceived by the subject by a second increased mental performance and the third perceived energy increase is perceived by the subject by a third increased mental performance after the second perceived energy increase, and wherein during the first perceived energy decline the second perceived energy increase become greater than the first perceived energy such that the subject perceives the second perceived energy increase prior to complete dissipation of the first perceived energy, and wherein during the second perceived energy decline the third perceived energy increase become greater than the second perceived energy such that the subject perceives the third perceived energy increase prior to complete dissipation of the second perceived energy.

In some embodiments, the energy composition, for example, may include a delivery system, an herb including Mormon tea, a methylxanthine including theobromine, and an active ingredient including phenylethylamine. The delivery system, for example, may be configured to deliver the active ingredient in an edible composition. For example, the medium may include edible solids, edible liquids, edible powders, liposomal creams, and/or any other delivery system configured to transfer chemicals into a body. In some embodiments, the energy composition may be administered through one or more of the delivery systems.

For example, edible solids may include: hard candy; soft candy; chewable candy; liquid capsulated pills; soft gel pills; chewable pills; multi-layered pills; dissolvable pills; effervescent pills configured to dissolve in liquids; lozenges; “gummy” candy; gelatin; edible food bars; dissolvable film; chewing gum; mints; frosting; edible coatings; frozen edible liquids; lip balm; and/or any other solid suitable for consumption. Embodiments including multi-layered pills, for example, may be configured such that each layer delivers a separate component of the energy composition.

For example, edible liquids may include: water; juice, including juice in concentrate; shakes; smoothies; edible gels; pastes, including semi-edible pastes such as toothpaste; mouthwash; vapors, such as, for example, humidifiers, aerosol sprays, breath sprays, nasal sprays, inhalers, electronic cigarette, and/or any other vapor suitable for inhalation; eye drops; condiments, such as sauces, syrup, honey, whipped cream and/or other condiment; and/or any other liquid or semi-liquid suitable for consumption. In some embodiments, the edible liquid may be frozen, for example, as an ice cube.

For example, edible powders may include: granulated edible solids; mixable powder configured to substantially dissolve into an edible liquid; powder configured to substantially mix with baking ingredients, such as, for example, baking powder, and/or sweetener; powder configured to be inhaled through the nasal cavity; edible candy powder; and/or any other edible powder suitable for consumption or substantial mixing with an edible liquid.

For example, liposomal creams may include: lotions; cosmetics; cosmetic creams; face wash; and/or any other liposomal cream suitable for substantial dermal absorption of chemicals.

The delivery system is not limited to only one category of delivery systems specified. In some embodiments, the delivery system, for example, may be composed of any number or combination of delivery systems, such as, for example, a mixable powder and edible liquid.

The herb may include plants, extracts from plants, or derivatives from extracts of plants. Such herbs may include, for example, Mormon tea, guayusa, and/or chaparro prieto. Herbs that include teas are not expressly limited to use with water, but may also include, for example, waterless compositions such as powders, leaves, and/or any other compositions that do not substantially use water. In some embodiments including chaparro prieto, chaparro prieto may be included, for example, in amounts from about 0.000018 g/mL to about 0.019 g/mL; and/or in amounts from about 0.00046 g/mL to about 0.0014 g/mL; and/or in amounts from about 0.0015 g/mL to about 0.0024 g/mL; and/or in amounts from about 0.0028 g/mL to about 0.0047 g/mL; and/or any other range suitable for use in an energy composition. In some embodiments including Mormon tea, Mormon tea may be included, for example, in amounts from about 0.000018 g/mL to about 0.019 g/mL; and/or in amounts from about 0.00037 g/mL to about 0.0015 g/mL; and/or in amounts from about 0.0015 g/mL to about 0.0024 g/mL; and/or in amounts from about 0.0025 g/mL to about 0.0037 g/mL; and/or in amounts from about 0.0038 g/mL to about 0.0047 g/mL; and/or in amounts from about 0.000082 g/mL to about 0.0047 g/mL; and/or any other range suitable for use in an energy composition. In some embodiments including guayusa, guayusa may be included, for example, in amounts from about 0.00018 g/mL to about 0.012 g/mL; and/or in amounts from about 0.0016 g/mL to about 0.0051 g/mL; and/or in amounts from about 0.0030 g/mL to about 0.0038 g/mL; and/or any other range suitable for use in an energy composition.

In some embodiments, the methylxanthine may include, for example, theobromine or caffeine. The methylxanthine may include plants and plant derivatives, such as the coffee bean, or synthesized, man-made derivatives. In some embodiments, the methylxanthine may include, for example, theobromine and caffeine. In some embodiments, theobromine may be included, for example, in amounts from about 0.000060 g/mL to about 0.012 g/mL; and/or in amounts from about 0.000094 g/mL to about 0.00028 g/mL; and/or in amounts from about 0.00030 g/mL to about 0.00094 g/mL; and/or in amounts from about 0.0010 g/mL to about 0.0019 g/mL; and/or in amounts from about 0.00083 g/mL to about 0.0012 g/mL; and/or in amounts from about 0.000060 g/mL to about 0.0019 g/mL; and/or any other range suitable for use in an energy composition.

In some embodiments, caffeine may be included, for example, in amounts from about 0.000018 g/mL to about 0.012 g/mL; and/or in amounts from about 0.00083 g/mL to about 0.012 g/mL; and/or in amounts from about 0.0028 g/mL to about 0.0050 g/mL; and/or in amounts from about 0.0030 g/mL to about 0.0038 g/mL; and/or in amounts from about 0.0035 g/mL to about 0.0057 g/mL; and/or any other range suitable for use in an energy composition.

Some embodiments may include, for example, one quantity of a methylxanthine substitute for another quantity of a separate methylxanthine. Some embodiments may include, for example, any combination of different quantities of methylxanthine. The methylxanthine may include natural sources such as cocoa, coffee beans, guarana etc. and other naturally occurring sources or extracts of naturally occurring sources. Methylxanthine may include caffeine, theobromine or man-made or synthesized sources derivatives or salts. In some embodiments including theobromine and caffeine, caffeine and theobromine may be included, for example, in amounts from about 0.0031 g/mL to about 0.0039 g/mL; and/or in amounts from about 0.0028 g/mL to about 0.0062 g/mL; and/or in amounts from about 0.00089 g/mL to about 0.014 g/mL; and/or any other suitable range for use in an energy composition.

The active ingredient may include, for example, phenylethylamine. Phenylethylamine may include natural sources such as cocoa, beans, lentils, nuts, seeds etc. and other naturally occurring sources or extracts of naturally occurring sources. Phenylethylamine may include man-made or synthesized sources such as phenylethylamine HCl and other salts or derivatives. In some embodiments, the active ingredient may also include one or more of the following: glucuronolactone, malic acid, taurine, yohimbine, and/or any other ingredient configured in a manner that may produce the perceived effects. For example, yohimbine may include natural sources such as the yohimbe plant and extracts of such sources. Yohimbine may include man-made or synthesized sources such as yohimbine HCl and other salts or derivatives. In some embodiments, the active ingredient may be included in any amount suitable for use in an energy composition.

In some embodiments including phenylethylamine, phenylethylamine may be included, for example, in amounts from about 0.000018 g/mL to about 0.010 g/mL; and/or in amounts from about 0.00094 g/mL to about 0.0018 g/mL; and/or in amounts from about 0.0018 g/mL to about 0.0042 g/mL; and/or in amounts from about 0.00094 g/mL to about 0.0083 g/mL; and/or in amounts from about 0.0030 g/mL to about 0.0094 g/mL; and/or any other range suitable for use in an energy composition.

In some embodiments including malic acid, malic acid may be included, for example, in amounts from about 0.00018 g/mL to about 0.034 g/mL; and/or in amounts from about 0.0094 g/mL to about 0.014 g/mL; and/or in amounts from about 0.0083 g/mL to about 0.034 g/mL; and/or in amounts from about 0.013 g/mL to about 0.016 g/mL; and/or in amounts from about 0.015 g/mL to about 0.038 g/mL; and/or any other range suitable for use in an energy composition.

In some embodiments including yohimbine, yohimbine may be included, for example, in amounts from about 0.000015 g/mL to about 0.00019 g/mL; and/or in amounts from about 0.000049 g/mL to about 0.00011 g/mL; and/or in amounts from about 0.000066 g/mL to about 0.000083 g/mL; and/or any other range suitable for use in an energy composition.

In some embodiments including glucuronolactone, glucuronolactone may be included, for example, in amounts from about 0.00018 g/mL to about 0.0076 g/mL; and/or in amounts from about 0.0033 g/mL to about 0.0042 g/mL; and/or in amounts from about 0.0018 g/mL to about 0.0057 g/mL; and/or any other range suitable for use in an energy composition.

In some embodiments including taurine, taurine may be included, for example, in amounts from about 0.00018 g/mL to about 0.029 g/mL; and/or in amounts from about 0.0094 g/mL to about 0.012 g/mL; and/or in amounts from about 0.013 g/mL to about 0.016 g/mL; and/or in amounts from about 0.015 g/mL to about 0.038 g/mL; and/or any other range suitable for use in an energy composition.

Some embodiments also may include, for example, one or more flavorants that enhance the palatability of the energy composition. Such flavorants may include, for example, acesulfame potassium, artificial strawberry, bitter blocker, citric acid, grape, N&A kiwi, N&A lemon-lime, N&A watermelon, natural bitter blocker, natural kiwi, natural strawberry, sodium chloride, sucralose, and/or any other flavorant suitable for improving palatability. One or more flavorants may be included in any number of quantities or combinations suitable for improving palatability.

Some embodiments also may include, for example, one or more preservatives configured to improve shelf life of the energy composition. Preservatives may include, for example, potassium sorbate, sodium benzoate, or any other preservative suitable for improving shelf life of an energy composition. The preservatives may be included in any combination or quantity suitable for improving the shelf life of the energy composition.

Some embodiments also may include, for example, one or more coloring agents configured to alter the color of the energy composition. Coloring agents may include, for example, blue lake #1, red lake #40, or any other coloring agent suitable for use in an energy composition. Coloring agents may be included in any quantity or combination configured to alter the color of the energy composition.

Some embodiments may include, for example, a gumming agent configured to provide a palatable texture, consistency, and/or enhance absorption of at least one flavorant. The gumming agent may include, for example, xanthan gam, cellulose gum, carrageenan gum, or any other gum suitable for use in an energy composition. In embodiments including a gumming agent, the gumming agent may be included, for example, in amounts from about 0.00018 g/mL to about 0.012 g/mL; or in amounts from about 0.00022 g/mL to about 0.00045 g/mL; or in amounts from about 0.00050 g/mL to about 0.00085 g/mL; or any other range suitable for use in an energy composition.

Some embodiments of the energy composition may include, for example, a delivery system and a combination of at least one herb, at least one methylxanthine, and at least one active ingredient. One or more herbs, one or more forms of methylxanthine, and one or more active ingredients may be combined in a manner suitable for producing the perceived effects of the energy composition. Such combinations may include, for example, any of the previously specified ranges of any of the herbs, forms of methylxanthine, and active ingredients. Additionally, such embodiments may include, for example, a gumming agent and one or more flavorants.

In some embodiments, a method of formulating an energy composition may comprise, for example, heating water to a temperature from about 80 to 100 degrees Celsius, adding Mormon tea in an amount from about 0.000082 g/mL to about 0.0047 g/mL to the water, creating an herb and water mixture. The herb and water mixture may be, for example, removed from the heat and soaked for no shorter than 30 minutes or until active constituents are released. The herb and water mixture may be, for example, mixed with a methylxanthine including theobromine in an amount from about 0.000083 g/mL to about 0.0012 g/mL and mixed with an active ingredient including phenylethylamine in an amount from about 0.00094 g/mL to about 0.010 g/mL. The energy composition may, for example, be formulated in a predetermined volume for consumption.

The method of formulating an energy composition may include, for example, adding caffeine in an amount from about 0.0028 g/mL to about 0.0050 g/mL. The method of formulating an energy composition may include, for example, adding caffeine and theobromine in an amount from about 0.0028 g/mL to about 0.0062 g/mL, coupled to stabilize the perceived effects. The method of formulating an energy composition may include, for example, adding malic acid in an amount from about 0.010 g/mL to about 0.014 g/mL. The method of formulating an energy composition may include, for example, adding yohimbine in an amount from about 0.000049 g/mL to about 0.000098 g/mL.

In some embodiments, an energy composition may comprise, for example, Mormon tea in an amount from about 0.00037 g/mL to about 0.0015 g/mL, theobromine and caffeine in an amount from about 0.0031 g/mL to about 0.0039 g/mL, and phenylethylamine in an amount from about 0.0018 g/mL to about 0.0042 g/mL.

The energy composition may further comprise, for example, water, wherein the Mormon tea is brewed in the water for no shorter than 30 minutes or until active constituents are released. The energy composition may comprise, for example, caffeine in an amount from about 0.0030 g/mL to about 0.0038 g/mL. The energy composition may comprise, for example, malic acid in an amount from about 0.010 g/mL to about 0.014 g/mL. The energy composition may comprise, for example, yohimbine in an amount from about 0.000049 g/mL to about 0.000097 g/mL.

In some embodiments, an energy composition may comprise, for example, an herb including Mormon tea in an amount from about 0.000082 g/mL to about 0.0047 g/mL, a methylxanthine including theobromine in an amount from about 0.000083 g/mL to about 0.0012 g/mL, and an active ingredient including phenylethylamine in an amount from about 0.00094 g/mL to about 0.0083 g/mL.

The energy composition may further comprise, for example, water, wherein the Mormon tea is brewed in the water for no shorter than 30 minutes or until active constituents are released. The energy composition may further comprise, for example, an herb including guayusa in an amount from about 0.00041 g/mL to about 0.0012 g/mL. The energy composition may further comprise, for example, a methylxanthine including caffeine in an amount from about 0.0028 g/mL to about 0.0050 g/mL. The energy composition may comprise, for example, caffeine and theobromine in an amount from about 0.0028 g/mL to about 0.0062 g/mL, coupled to stabilize the perceived effects. The energy composition may comprise, for example, an active ingredient including malic acid in an amount from about 0.011 g/mL to about 0.014 g/mL. The energy composition may comprise, for example, an active ingredient including yohimbine in an amount from about 0.000066 g/mL to about 0.000083 g/mL.

In some embodiments, an energy composition may comprise, for example, Mormon tea in an amount from about 0.000018 g/mL to about 0.019 g/mL, a methylxanthine including theobromine in an amount from about 0.000060 g/mL to about 0.0019 g/mL and caffeine in an amount from about 0.00083 g/mL to about 0.012 g/mL, and an active ingredient including phenylethylamine in an amount from about 0.000018 g/mL to about 0.010 g/mL.

The energy composition may further comprise, for example, water, wherein the Mormon tea is brewed in the water for no shorter than 30 minutes or until active constituents are released. The energy composition may comprise, for example, an active ingredient including malic acid in an amount from about 0.0083 g/mL to about 0.034 g/mL. The energy composition may comprise, for example, an active ingredient including yohimbine in an amount from about 0.000015 g/mL to about 0.00017 g/mL.

The following tables provide a few examples of an energy composition according to some embodiments. In the examples provided in the following tables, the listed ingredients may be combined with the delivery system. In embodiments where the energy composition is an edible liquid, the delivery system may be configured as an edible liquid, such as, for example, water. In these embodiments, the sum of the listed ingredients and the edible liquid may have a total volume of about 53 mL.

In some embodiments, such as, for example, that illustrated in Table 1, the energy composition may include, for example, 25 mg to 75 mg of Mormon tea. Mormon tea may be, for example, added to water that is between 80 and 100 degrees Celsius. Mormon tea may be brewed in the heated water for no less than 30 minutes and/or until the active constituents are released. Mormon tea may produce the perceived effects almost immediately after ingestion, wherein Mormon tea may take effect, for example, in about 5 to 10 minutes and wherein the term “immediately” means between 5 and 25 minutes. The half-life of Mormon tea, for example, is about 15 to 20 minutes. The perceived effects of Mormon tea may be increasingly strong effect proportional to the amount of time Mormon tea is brewed.

In some embodiments, the energy composition may also include, for example, 190 mg to 210 mg of caffeine and 5 mg to 15 mg of theobromine. Caffeine may take effect, for example, about 30 to 40 minutes after ingestion, and caffeine has a half-life, for example, of about 2.5 to 5 hours. Theobromine may take effect, for example, about 2 to 3 hours after ingestion, and theobromine has a half-life, for example, of about 7 to 12 hours. The combination of caffeine and theobromine, for example, may create longer lasting perceived effects. The addition of both caffeine and theobromine, for example, may reduce the potential for a “sugar crash” or “caffeine crash.” For example, theobromine may take effect as the caffeine reaches its half-life. Additionally, the onset of the perceived effects of theobromine, although more potent, may be significantly slower than caffeine. Thus, the addition of theobromine may, for example, prevent a “sugar crash” or “caffeine crash.”

In some embodiments, the energy composition may also include, for example, 110 mg to 146 mg of phenylethylamine. Phenylethylamine may, for example, increase cognitive enhancement and mood improvement, and/or act as a concentration aid. Phenylethylamine may, for example, react with Mormon tea in a manner that may create the perceived effects immediately. Phenylethylamine may create the perceived effects of natural energy and heightened mental concentration.

In some embodiments, the energy composition may include, for example, 180 mg to 220 mg of glucuronolactone. The energy composition may also include, for example, 700 mg to 800 mg of malic acid. The energy composition may include, for example, 700 mg to 800 mg of taurine. The energy composition may include, for example 4 mg to 5 mg of yohimbine.

In some embodiments, the energy composition may include, for example, any combination of herbs, methylxanthines, and active ingredients. Additionally, such embodiments may include a gumming agent and/or one or more flavorants. In some embodiment, the energy composition may include, for example, 27 mg to 45 mg of gumming agent and 1300 mg to 1400 mg of flavorants.

TABLE 1

| Component | Amount (mg) |
|---|---|
| Caffeine | 190-210 |
| Mormon tea | 25-75 |
| Glucuronolactone | 180-220 |
| Malic Acid | 700-800 |
| Phenylethylamine | 110-146 |
| Taurine | 700-800 |
| Theobromine | 5-15 |
| Yohimbine | 4-5 |
| Gumming Agent | 27-45 |
| Preservatives | 50-60 |
| Flavorants | 1300-1400 |
| Color | 15-25 |

In some embodiments, such as, for example, that illustrated in Table 2, the energy composition may include, for example, 35 mg to 85 mg of Mormon tea. Mormon tea takes effect, for example, in about 5 to 10 minutes. The half-life of Mormon tea, for example, is about 15 to 20 minutes. The perceived effects of Mormon tea may be increasingly strong proportional to the amount of time Mormon tea is brewed. Additionally, the perceived effects may be a stronger when the amount of Mormon tea is increased in the energy composition.

In some embodiments, the energy composition may also include, for example, 60 mg to 260 mg of caffeine and 10 mg to 50 mg of theobromine. Including theobromine may prevent a “sugar crash” or “caffeine crash.” For example, theobromine may take effect as caffeine reaches its half-life, which may reduce the potential for a “sugar crash” or “caffeine crash.” Theobromine's slow onset and long lasting perceived effects, for example, may counter caffeine's rapid onset and dissipation. Additionally, the perceived effects of theobromine may be mild in comparison to caffeine, which may create a slow taper as the perceived effects of theobromine wear off.

In some embodiments, the energy composition may also include, for example, 100 mg to 250 mg of phenylethylamine. Phenylethylamine, for example, may react with Mormon tea in a manner that may create the perceived effects immediately. Phenylethylamine may create the perceived effects of natural energy and heightened mental concentration.

In some embodiments, the energy composition may include, for example, 100 mg to 300 mg of glucuronolactone. The energy composition may also include, for example, 340 mg to 1060 mg of malic acid. The energy composition may include, for example, 375 mg to 1170 mg of taurine. The energy composition may include, for example, 3 mg to 6 mg of yohimbine.

In some embodiments, the energy composition may include, for example, any combination of herbs, methylxanthines, and active ingredients. Additionally, such embodiments may include a gumming agent and/or one or more flavorants. In some embodiments, the energy composition may include, for example, 27 mg to 45 mg of gumming agent and 1300 mg to 1400 mg of flavorants.

TABLE 2

| Component | Amount (mg) |
|---|---|
| Caffeine | 60-260 |
| Mormon tea | 35-85 |
| Glucuronolactone | 100-300 |
| Malic Acid | 340-1060 |
| Phenylethylamine | 100-250 |
| Taurine | 375-1170 |
| Theobromine | 10-50 |
| Yohimbine | 3-6 |
| Gumming Agent | 27-45 |
| Preservatives | 50-60 |
| Flavorants | 1300-1400 |
| Color | 15-25 |

In some embodiments, such as, for example, that illustrated in Table 3, the energy composition may include, for example, 5 mg to 250 mg of Mormon tea. Mormon tea may be, for example, added to water that is between 80 and 100 degrees Celsius. Mormon tea may be brewed in the heated water for no less than 30 minutes and/or until the active constituents are released. Mormon tea may take effect, for example, in about 5 to 10 minutes. The half-life of Mormon tea, for example, is about 15 to 20 minutes. The perceived effects of Mormon tea may be increasingly strong proportional to the amount of time Mormon tea is brewed. Additionally, the perceived effects may be stronger when the amount of Mormon tea is increased in the energy composition.

In some embodiments, the energy composition may include, for example, 150 mg to 300 mg of caffeine and 5 mg to 100 mg of theobromine. The combination of caffeine and theobromine, for example, may create longer lasting perceived effects. The addition of both caffeine and theobromine, for example, may reduce the potential for a “sugar crash” or “caffeine crash.” Theobromine takes effect as the caffeine reaches its half-life. Additionally, the perceived effects of theobromine, although more potent, are significantly slower than caffeine. Thus, the addition of theobromine may prevent a “sugar crash” or “caffeine crash.”

In some embodiments, the energy composition may also include, for example, 80 mg to 500 mg of phenylethylamine. Phenylethylamine, for example, may react with Mormon tea in a manner that may create the perceived effects immediately. Phenylethylamine may create the perceived effects of natural energy and heightened mental concentration.

In some embodiments, the energy composition may include, for example, 100 mg to 300 mg of glucuronolactone. The energy composition may also include, for example, 500 mg to 2000 mg of malic acid. The energy composition may include, for example, 500 mg to 2000 mg of taurine. The energy composition may include, for example 1 mg to 10 mg of yohimbine.

In some embodiments, the energy composition may include, for example, any combination of herbs, methylxanthines, and active ingredients. Additionally, such embodiments may include a gumming agent and/or one or more flavorants. In some embodiments, the energy composition may include, for example, 10 mg to 600 mg of gumming agent and 750 mg to 2700 mg of flavorants.

TABLE 3

| Component | Amount (mg) |
|---|---|
| Caffeine | 150-300 |
| Mormon tea | 5-250 |
| Glucuronolactone | 100-300 |
| Malic Acid | 500-2000 |
| Phenylethylamine | 80-500 |
| Taurine | 500-2000 |
| Theobromine | 5-100 |
| Yohimbine | 1-10 |
| Gumming Agent | 10-600 |
| Preservatives | 50-70 |
| Flavorants | 750-2700 |
| Color | 5-25 |

In some embodiments, such as, for example, that illustrated in Table 4, the energy composition may include 1 mg to 1000 mg of Mormon tea. Mormon tea may take effect, for example, about 5 to 10 minutes after ingestion. The half-life of Mormon tea, for example, is about 15 to 20 minutes. The perceived effects of Mormon tea may be increasingly strong proportional to the amount of time Mormon tea is brewed. Additionally, the perceived effects may be stronger when the amount of Mormon tea is increased in the energy composition.

In some embodiments, the energy composition may also include, for example, 10 mg to 600 mg of caffeine and 10 mg to 600 mg of theobromine. Including theobromine may reduce the potential for a “sugar crash” or “caffeine crash.” For example, theobromine may take effect as caffeine reaches its half-life, which may reduce the potential for a “sugar crash” or “caffeine crash.” Theobromine's slow onset and long lasting perceived effects, for example, may counter caffeine's rapid onset and dissipation. Additionally, the perceived effects of theobromine may be mild in comparison to caffeine, which may create a slow taper as the perceived effects of theobromine wear off.

In some embodiments, the energy composition may also include, for example, 1 mg to 500 mg of phenylethylamine. Phenylethylamine, for example, may react with Mormon tea in a manner that may create the perceived effects immediately. Phenylethylamine may create the effects of natural energy and heightened mental concentration.

In some embodiments, the energy composition may include, for example, 10 mg to 400 mg of glucuronolactone. The energy composition may also include, for example, 10 mg to 1500 mg of malic acid. The energy composition may include, for example, 10 mg to 1500 mg of taurine. The energy composition may include, for example 0.05 mg to 15 mg of yohimbine.

In some embodiments, the energy composition may include, for example, any combination of herbs, methylxanthines, and active ingredients. Additionally, such embodiments may include a gumming agent and/or one or more flavorants. In some embodiments, the energy composition may include, for example, 10 mg to 600 mg of gumming agent and 750 mg to 2700 mg of flavorants.

TABLE 4

| Component | Amount (mg) |
|---|---|
| Caffeine | 10-600 |
| Mormon tea | 1-1000 |
| Guayusa | 10-600 |
| Glucuronolactone | 10-400 |
| Malic Acid | 10-1500 |
| Phenylethylamine | 1-500 |
| Taurine | 10-1500 |
| Theobromine | 10-600 |
| Yohimbine | 0.05-15 |
| Gumming Agent | 10-600 |
| Preservatives | 50-70 |
| Flavorants | 750-2700 |
| Color | 5-25 |

TABLE 5

| Component | Amount (mg) |
|---|---|
| Caffeine | 60-2000 |
| Mormon tea | 35-1000 |
| Glucuronolactone | 100-500 |
| Malic Acid | 340-1060 |
| Phenylethylamine | 100-2000 |
| Taurine | 375-1170 |
| Theobromine | 10-150 |
| Yohimbine | 3-6 |
| Gumming Agent | 27-45 |
| Preservatives | 50-60 |
| Flavorants | 1300-1400 |
| Color | 15-25 |

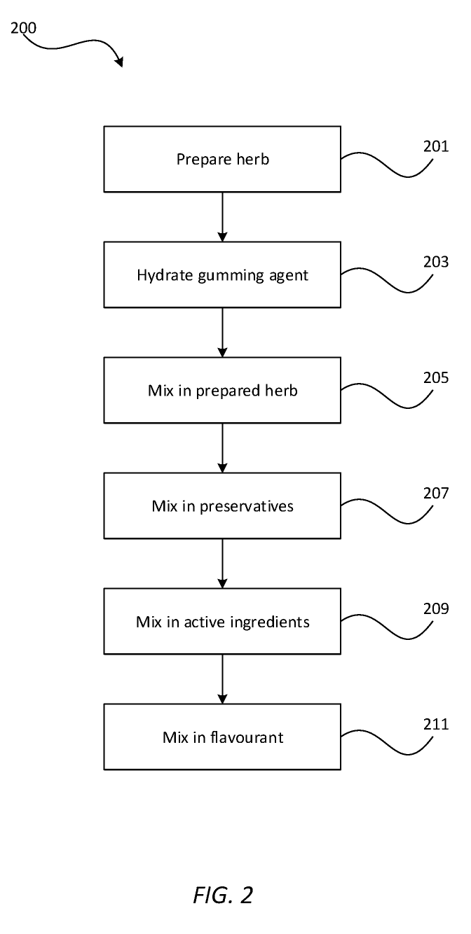

FIG. 2 illustrates a block diagram for an example method 200 for preparing some embodiments of an energy composition. The blocks shown in FIG. 2 can be rearranged or removed from method 200. Additional blocks, steps, process, and/or methods, etc. may be added to method 200.

Method 200 may include block 201, block 203, block 205, block 207, block 209, and block 211. Example embodiments may include mixing, which may include, for example, mixing through a high sheer mixer or another type of mixer. The high sheer mixer may include, for example, any method for substantially mixing two or more components, such as, for example, a high speed blender, a miller, a grinder, and/or any other method suitable for substantially mixing two or more ingredients.

Block 201 may include preparing one or more herbs to be included in the energy composition. Herbs that include teas are not expressly limited to use with water, but may also include, for example, waterless compositions such as powders, leaves, and/or any other compositions that do not substantially use water. Preparing the herbs may include, for example, altering the state of the herb in a manner suitable for mixing with one or more of the delivery system, active ingredient, methylxanthine, gumming agent, preservative, coloring, and/or flavorant, etc. For example, such preparation may include shredding, crushing, dicing, burning, pulverizing, and/or any other manner suitable for mixing with one or more of the delivery system, active ingredient, methylated xanthine, gumming agent, preservative, coloring, and/or flavorant, etc. Preparing the herbs may include any method for extracting components from the herbs into a form suitable for mixing with one or more of the delivery system, active ingredient, methylxanthine, gumming agent, preservative, coloring, and/or flavorant. Such preparing may include forms such as, for example, steeping, brewing, boiling, steaming, mixing, and/or any other preparation method suitable for extracting chemicals from herbs. The herb may be prepared in a manner and for a time such that substantially all and/or some components of the herb, or herbs, may be extracted.

In some embodiments, the preparation may be done in a manner suitable for increasing the bio-availability of the extracted components. In some embodiments, the components of the herb may be held naturally in a bound matrix, and the preparation may be done in a manner suitable for releasing the extracted components from the bound matrix. Increasing the bio-availability of the extracted components may increase the rate at which the energy composition provides the perceived effects. Such preparation may include, for example, brewing the herb, or herbs, for a suitable time and in a suitable temperature. In some embodiments, the preparation may include, for example, using only the extracted components. In some embodiments, the herb and extracted components may be used.

In some embodiments, preparation of the herb may include, for example, heating water to a temperature between 80 and 100 degrees Celsius, adding the Mormon tea to the water to create an herb and water mixture, removing the herb and water mixture from the heat, and soaking the herb and water mixture for no shorter than 30 minutes and/or until the active constituents are released. In some embodiments, the water, for example, is heated to at least 80 degrees, but the water is not brought to a boil.

In some embodiments, a stainless steel mesh basket, for example, may be used to add the herb to the water. For example, the stainless steel mesh basket may include mesh of about 5 μm. Filters may also be used. However, in a large production, filters may cause possible problems with allowing water to completely saturate the herb. Using the stainless steel mesh basket, for example, may allow water to substantially or completely saturate the herb.

Brewing the herb may create a more effective energy composition. For example, brewing the herb may, for example, eliminate sedimentation. Brewing the herb may also provide a more immediate effect of the energy composition. Additionally, the effect of the herb strengthens relative to the length of time the herb is brewed.

Block 203 may include, for example, hydrating the gumming agent. Hydration may include any method for substantially hydrating the gumming agent to about 10 parts water to 1 part gumming agent. Some embodiments may include, for example, mixing the gumming agent with water. Hydration of the gumming agent may include various mixing times to mix the gumming agent with water. Some embodiments may include a hydration time, for example, of about seven to ten minutes.

Block 205 may include mixing the prepared herbs with the hydrated gumming agent. Block 207 may include mixing the preservatives with the mixed herbs and hydrated gumming agent. Block 209 may include mixing the active ingredients with the mixed herbs, hydrated gumming agent, and preservatives. In some embodiments, the order may include mixing active ingredients with a lower dissolvability before mixing active ingredients with a higher dissolvability. For example, block 209 may include mixing taurine before other active ingredients. Block 211 may include mixing the flavorant and/or flavorants with the mixed herbs, hydrated gumming agent, preservatives, and active ingredients. In some embodiments, the order of mixing blocks 205, 207, 209, and 211 may vary. For example, the active ingredients may be mixed with the prepared herb before the hydrated gumming agent and/or preservatives are added to the solution.

Embodiments of the method 200 that include mixing may include various mixing times configured to combine the components of the energy composition into a suitable form that may provide the perceived effects of an energy composition.

Modifications may be made to method 200 without changing the scope of the disclosure. In some embodiments, block 203 may include, for example, hydrating the gumming agent with the preservatives. Method 200 may also omit any block in which the corresponding component is not included in the composition. For example, embodiments omitting the gumming agent may omit block 203; embodiments omitting active ingredients may omit block 209.

Each component used herein may have a different name associated with such a component. For example, Mormon tea may be referred to as ephedra viridis or Brigham tea. Ephedra viridis is a species of the ephedra genus. There may be up to 36 different plant species of ephedra, many of which may include weight loss or fat burning properties.

In some embodiments, Ephedra viridis, or Mormon tea, does not contain ephedrine alkaloids such as, for example, ephedrine alkaloids that have been banned by the FDA. Ephedra viridis may include thermogenic properties and energy inducing properties and without, for example, the undesired side effects caused by ephedra sinica.

The addition of theobromine manages the bioavailability of caffeine. Theobromine is a metabolite of caffeine. The addition of theobromine may maintain the alkaloid effect of the energy composition for a longer period of time.

In some embodiments, an active constituent is not the same as active ingredient. Active constituents, for example, may be released when an herb is heated. In addition, active constituents, for example, may be released from the herb that may help increase the perceived effects. An active ingredient, for example, may be a separate ingredient that may be added to the energy composition.

It should be recognized that the ingredients of the compositions described herein can be the exact ingredients or biological equivalents. Also, reference to an ingredient, such as caffeine, also references any extract having the caffeine and any salt, polymorph, other form, or the like of that ingredient. This applies to all ingredients. Also, reference to an ingredient such as yohimbine HCl also refers to yohimbine alone without HCL or with some other salt, acidic or basic form, or other derivatives thereof, and reference to yohimbine can include yohimbine HCL. Additionally, reference to an ingredient such as phenylethylamine HCl also refers to phenylethylamine alone without HCL or with some other salt, acidic or basic form, or other derivatives thereof, and reference to phenylethylamine can include phenylethylamine HCl.

The term “substantially” means within 5% or 10% of the value referred to or within manufacturing tolerances.

Various embodiments are disclosed. The various embodiments may be partially or completely combined to produce other embodiments.

All examples and conditional language recited herein are intended for pedagogical objects to aid the reader in understanding the present invention and the concepts contributed by the inventor to furthering the art, and are to be construed as being without limitation to such specifically recited examples and conditions. Although embodiments of the present invention have been described in detail, it should be understood that the various changes, substitutions, and alterations may be made hereto without departing from the spirit and scope of the present invention.

What is claimed is:

1. A method for increasing energy in a subject, the method comprising:
providing the subject, wherein the subject has an initial perceived energy level, and wherein the subject has a desire for an increased perceived energy level that is greater than the initial perceived energy level; and
administering a composition to the subject, the composition having Mormon tea in an amount from about 0.00037 g/mL to about 0.0015 g/mL that provides a first perceived energy and a first methylxanthine in an amount from about 0.0030 g/mL to about 0.0038 g/mL that provides a second perceived energy and a second methylxanthine in an amount from about 0.000083 g/mL to about 0.0012 g/mL that provides a third perceived energy, wherein:
the composition provides the first perceived energy having a first perceived energy increase within about 10 minutes of administering the composition from the herb including Mormon tea, wherein then the first perceived energy has a first perceived energy peak and then a first perceived energy decline;
the composition provides the second perceived energy having a second perceived energy increase within about 40 minutes of administering the composition from the first methylxanthine, wherein then the second perceived energy has a second perceived energy peak and then a second perceived energy decline;
the composition provides the third perceived energy having a second perceived energy increase within about 135 minutes of administering the composition from the second methylxanthine, wherein then the third perceived energy has a third perceived energy peak and then a third perceived energy decline;
wherein the first perceived energy increase is perceived by the subject before the second perceived energy increase and the third perceived energy increase is perceived by the subject after the second perceived energy increase, and wherein during the first perceived energy decline the second perceived energy increase become greater than the first perceived energy such that the subject perceives the second perceived energy increase prior to complete dissipation of the first perceived energy, and wherein during the second perceived energy decline the third perceived energy increase become greater than the second perceived energy such that the subject perceives the third perceived energy increase prior to complete dissipation of the second perceived energy.

2. The method of claim 1, wherein the first methylxanthine includes caffeine and the second methylxanthine includes theobromine.

3. The method of claim 1, wherein the composition includes phenylethylamine in amount from about 0.0018 g/mL to about 0.0042 g/mL that contributes to providing the first perceived energy.

4. The method of claim 1, wherein the composition includes yohimbine in an amount from about 0.000049 g/mL to about 0.000097 g/mL that contributes to providing the first perceived energy.

5. The method of claim 1, wherein the composition includes phenylethylamine in amount from about 0.0018 g/mL to about 0.0042 g/mL and yohimbine in an amount from about 0.000049 g/mL to about 0.000097 g/mL, wherein the phenylethylamine and yohimbine both contribute to providing the first perceived energy.

6. The method of claim 1, wherein at least one of the Mormon tea, first methylxanthine or second methylxanthine is provided in the composition as an extract of a plant.

7. The method of claim 1, wherein the composition includes malic acid in an amount from about 0.010 g/mL to about 0.014 g/mL.

8. The method of claim 1, wherein the composition includes guayusa in an amount from about 0.00041 g/mL to about 0.012 g/mL that contributes to providing the second perceived energy.

9. The method of claim 1, wherein the herb including Mormon tea is brewed in water for no shorter than 30 minutes or until active constituents are released before being formulated into the composition.

10. The method of claim 1, wherein prior to administering the composition, the subject has an initial alertness level, and wherein the subject has a desire for an increased alertness level that is greater than the initial alertness level, wherein the first perceived energy, second perceived energy, and third perceived energy provide the increased alertness.

11. The method of claim 1, wherein prior to administering the composition, the subject feels fatigued, wherein the first perceived energy, second perceived energy, and third perceived energy reduce the feeling of being fatigued.

12. The method of claim 1, wherein prior to administering the composition, the subject has an initial mental concentration level, and wherein the subject has a desire for an increased mental concentration level that is greater than the initial alertness level, wherein the first perceived energy, second perceived energy, and third perceived energy provide the increased mental concentration.

13. The method of claim 1, wherein the subject does not perceive energy lower than the initial perceived energy level until after complete dissipation of the third perceived energy.

14. The method of claim 1, wherein the subject does not feel a need to increase their perceived energy level until during or after the third perceived energy decline.

15. The method of claim 1, wherein the administering of the composition is in an oral bolus consumed orally substantially within about 1 second, or within about 2 seconds, or within about 3 seconds, or within about 4 seconds, or within about 5 seconds, or within about 10 seconds, or within about 20 seconds, or within about 30 seconds, or within about 1 minute.

16. The method of claim 1, wherein the composition is a liquid beverage that is administered orally.

17. The method of claim 1, wherein the subject does not perceive an energy crash until after complete dissipation of the third perceived energy.

18. A method for increasing energy in a subject, the method comprising:
providing the subject, wherein the subject has an initial perceived energy level, and wherein the subject has a desire for an increased perceived energy level that is greater than the initial perceived energy level, and wherein the subject has a desire for sustained perceived energy; and
administering a composition to the subject, the composition having:
Mormon tea in an amount from about 0.00037 g/mL to about 0.0015 g/mL and phenylethylamine in amount from about 0.0018 g/mL to about 0.0042 g/mL, the Mormon tea and phenylethylamine cooperatively provides a first perceived energy;
a first methylxanthine including caffeine in an amount from about 0.0030 g/mL to about 0.0038 g/mL that provides a second perceived energy; and
a second methylxanthine including theobromine in an amount from about 0.000083 g/mL to about 0.0012 g/mL that provides a third perceived energy, wherein:
the composition provides the first perceived energy having a first perceived energy increase within about 10 minutes of administering the composition from the herb including Mormon tea, wherein then the first perceived energy has a first perceived energy peak and then a first perceived energy decline;
the composition provides the second perceived energy having a second perceived energy increase within about 40 minutes of administering the composition from the first methylxanthine, wherein then the second perceived energy has a second perceived energy peak and then a second perceived energy decline;
the composition provides the third perceived energy having a second perceived energy increase within about 135 minutes of administering the composition from the second methylxanthine, wherein then the third perceived energy has a third perceived energy peak and then a third perceived energy decline;
wherein the first perceived energy increase is perceived by the subject before the second perceived energy increase and the third perceived energy increase is perceived by the subject after the second perceived energy increase, and wherein during the first perceived energy decline the second perceived energy increase become greater than the first perceived energy such that the subject perceives the second perceived energy increase prior to complete dissipation of the first perceived energy, and wherein during the second perceived energy decline the third perceived energy increase become greater than the second perceived energy such that the subject perceives the third perceived energy increase prior to complete dissipation of the second perceived energy so as to provide the sustained perceived energy.

19. The method of claim 18, wherein the composition includes yohimbine in an amount from about 0.000049 g/mL to about 0.000097 g/mL that contributes to providing the first perceived energy.

20. A method for increasing mental performance in a subject, the method comprising:
providing the subject, wherein the subject has an initial perceived energy level for an initial mental performance, and wherein the subject has a desire for an increased perceived energy level for an increased mental performance that is greater than the initial mental performance; and
administering a composition to the subject, the composition having:
Mormon tea in an amount from about 0.00037 g/mL to about 0.0015 g/mL and phenylethylamine in amount from about 0.0018 g/mL to about 0.0042 g/mL, the Mormon tea and phenylethylamine cooperatively provides a first perceived energy;
a first methylxanthine including caffeine in an amount from about 0.0030 g/mL to about 0.0038 g/mL that provides a second perceived energy; and
a second methylxanthine including theobromine in an amount from about 0.000083 g/mL to about 0.0012 g/mL that provides a third perceived energy, wherein:
the composition provides the first perceived energy having a first perceived energy increase within about 10 minutes of administering the composition from the herb including Mormon tea, wherein then the first perceived energy has a first perceived energy peak and then a first perceived energy decline, wherein the first perceived energy correlates with a first mental performance;
the composition provides the second perceived energy having a second perceived energy increase within about 40 minutes of administering the composition from the first methylxanthine, wherein then the second perceived energy has a second perceived energy peak and then a second perceived energy decline, wherein the second perceived energy correlates with a second mental performance;
the composition provides the third perceived energy having a second perceived energy increase within about 135 minutes of administering the composition from the second methylxanthine, wherein then the third perceived energy has a third perceived energy peak and then a third perceived energy decline, wherein the third perceived energy correlates with a third mental performance;
wherein the first perceived energy increase is perceived by the subject by increased mental performance before the second perceived energy increase is perceived by the subject by a second increased mental performance and the third perceived energy increase is perceived by the subject by a third increased mental performance after the second perceived energy increase, and wherein during the first perceived energy decline the second perceived energy increase become greater than the first perceived energy such that the subject perceives the second perceived energy increase prior to complete dissipation of the first perceived energy, and wherein during the second perceived energy decline the third perceived energy increase become greater than the second perceived energy such that the subject perceives the third perceived energy increase prior to complete dissipation of the second perceived energy.
